(19) United States
(12) Patent Application Publication
(10) Pub. No.: US 2025/0241770 A1
(43) Pub. Date: Jul. 31, 2025

(54) AMPUTATION ENDCAP DEVICE FOR PREVENTION OF BONE OVERGROWTH

(71) Applicant: Ryan Miller, Gilbert, AZ (US)

(72) Inventor: Ryan Miller, Gilbert, AZ (US)

(21) Appl. No.: 19/037,923

(22) Filed: Jan. 27, 2025

Related U.S. Application Data
(60) Provisional application No. 63/625,393, filed on Jan. 26, 2024.

(51) Int. Cl. A61F 2/78

(52) U.S. Cl. CPC A61F 2/7812

(57) ABSTRACT

An prosthetic endcap device for amputated bone comprises an annular body with an expansion shield and integrated fixation elements. The device features a trabeculated surface for tissue integration, multiple transosseous holes for screw fixation, and an intramedullary hole for bone attachment. The expansion shield extends 5-10 mm radially with a bulbous profile at 10-45 degrees. Key dimensions include 3-5 mm body thickness, 15-40 mm height, and 1-2 mm diameter transosseous holes. Construction uses biocompatible materials including titanium alloys and PEEK. The device can connect to a second endcap via adjustable rods for dual-bone applications. Additional features include radiopaque markers, longitudinal grooves for bone ingrowth, and myodesis holes for soft tissue attachment. This innovation addresses amputation challenges with adaptable fixation and growth accommodation.

### CROSS REFERENCE TO RELATED APPLICATIONS

This application claims benefit from currently pending U.S. Provisional Application No. 63/625,393 titled “Pediatric Amputation Endcap for Prevention of Bone Overgrowth” and having a filing date of Jan. 26, 2024, all of which is incorporated by reference herein.

### FIELD OF THE INVENTION

The present invention relates generally to the field of prosthetic devices, and more particularly to adult, pediatric or any animal prosthetic components and even more specifically to a prosthetic endcap device designed for use in adult, pediatric, or any animal limb amputations. The invention further relates to devices and methods for managing soft tissue attachment, promoting optimal tissue integration, and accommodating skeletal growth in adult, pediatric or any animal amputees.

### BACKGROUND OF THE INVENTION

Adult, pediatric or animal amputations of both upper and lower extremities present unique challenges in prosthetic management due to continued skeletal growth and development. Traditional amputation techniques and prosthetic solutions designed for adults often fail to address the specific needs of adult and pediatric patients, particularly in terms of accommodating growth, maintaining bone health, and ensuring optimal functional outcomes across different types of long bone amputations.

The management of terminal bone overgrowth in amputations, particularly of the humerus and tibia or any limb, has been a persistent challenge in the field. Historical approaches have centered around two main strategies: the use of autogenous bone grafts and endoprosthetic caps. While these methods have helped avoid frequent stump revisions and re-amputations, each presents its own limitations. Autogenous bone grafts are often constrained by graft availability, require multiple surgical incisions, and necessitate subsequent removal of fixation metals, creating additional surgical burden on young patients.

A significant advancement came in 1985 with the Marquardt endoprosthetic stump cap, developed in collaboration with Hannover and the MECRON Company. This device, consisting of a titanium body and a polyethylene head, represented a notable improvement by allowing early weight-bearing and enabling the use of a definitive prosthesis within weeks of the procedure. However, the long-term efficacy of this approach remains uncertain due to limited longitudinal data and unknown complication rates.

The preservation of growth plates plays a crucial role in determining the final length of the residual limb in adult and pediatric patients. When these growth plates are removed, it can result in a significantly shorter final stump and potential bone overgrowth at the transected end. This overgrowth pattern may persist until growth completion, often necessitating multiple trimming procedures throughout development. Furthermore, in cases involving paired bones such as the tibia-fibula complex, disparate growth rates can lead to complications like the formation of sharp bone spurs that gradually penetrate the overlying soft tissues.

Procedures developed by pioneers like Swanson and Marquardt have attempted to transform diaphyseal deficiencies or amputations into stumps that mirror disarticulation types. While these approaches have advanced the field, current solutions still face several limitations, including inadequate soft tissue management, difficulty in maintaining proper muscle tension, and challenges in promoting healthy bone growth patterns. These issues can lead to complications such as bone overgrowth, soft tissue redundancy, and reduced functional capacity of the residual limb, regardless of whether the amputation involves upper or lower extremities.

Existing prosthetic endcap devices typically lack features specifically designed for adult and pediatric applications across different anatomical locations. The absence of proper scaling relative to adult and pediatric anatomy can result in poor fit and compromised function in both arm and leg amputations. Additionally, conventional devices often fail to provide adequate attachment points for soft tissue stabilization and may not incorporate features to promote optimal tissue integration, issues that affect all long bone amputations in children.

The interface between bone and soft tissue presents another critical consideration in pediatric amputations of both upper and lower extremities. Current solutions often lack adequate features for promoting proper tissue attachment and growth, which can lead to poor outcomes in terms of prosthetic fit and function. The absence of properly designed surfaces for tissue integration can result in reduced stability and compromised biomechanical function of the residual limb, regardless of amputation location.

Therefore, there exists a need for a prosthetic endcap device specifically designed amputations that addresses these challenges while accommodating the unique anatomical and physiological requirements of growing children across different amputation sites. Such a device should provide proper soft tissue management, secure fixation options, and features that promote optimal tissue integration while maintaining the ability to adapt to the growing pediatric patient, whether applied to upper or lower extremity amputations.

### SUMMARY OF THE INVENTION

The current invention provides for a prosthetic endcap device for adult, pediatric or any animal amputations of a patient having an amputated bone having a bone end comprising an annular body with an axis, interior, proximal end for the bone end, and a distal end with a bottom. The device features an expansion shield extending outward from the axis at the distal end. A plurality of transosseous holes in the annular body accommodate transosseous screws. The bottom includes an intramedullary hole for an intramedullary screw, plus interior and exterior surfaces. Either surface can be trabeculated with peaks and valleys of 1-2 mm height or depth. The expansion shield can extend 5-10 mm radially beyond the trabeculated surface. The annular body can measures 3-5 mm thick and 20% of the total device height.

The transosseous holes can measure 1-2 mm in diameter and encircle the periphery wherein the transosseous holes can be arranged in at least one circular pattern at different heights along the annular body 20. The intramedullary hole can fit a 2-6 mm diameter screw that fastens to the amputated bone end. The annular body can allow for either press or loose fitting onto the bone. Transosseous holes can avoid the central intramedullary canal. Construction materials of the device can include PEEK, nylon, carbon fiber, titanium, titanium alloys, and stainless steel. The expansion shield can feature a bulbous shape and extends 10-45 degrees from the axis. The annular body height ranges from such as, for example, at least 10 millimeters to 60 millimeters, and more preferable 12 millimeters to 50 millimeters and still even more preferable 15 millimeters to 40 millimeters.

A second endcap device may be provided that is connected to the endcap device by an customizable connection rod to secure adjacent amputated bones in position relative to each other. The connection rod may be telescoping to adjust during placement of the endcaps or may be fixed prior to placement in a patient. This second device can be smaller than the endcap device and can be coupled to the fibula in case of the tibia amputation or radius in case of the ulna amputation. The transosseous holes form multiple circular patterns at varying heights. Additional features include radiopaque markers at specific locations, longitudinal grooves for bone ingrowth on the interior surface, and myodesis holes for suture attachment. The prosthetic endcap device can address the unique challenges of adult and pediatric amputations by providing stable fixation, promoting bone growth, and accommodating soft tissue management.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors' intent and desire that the simple, plain and ordinary meaning to the terms be applied to the interpretation of the specification and claims. Aspects and applications of the invention presented here are described below in the drawings and detailed description of the invention.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112 (f). Thus, the use of the words “function,” “means” or “step” in the Detailed Description or Description of the Drawings or claims is not intended to somehow indicate a desire to invoke the special provisions of 35 U.S.C. § 112 (f), to define the invention. To the contrary, if the provisions of 35 U.S.C. § 112 (f) are sought to be invoked to define the inventions, the claims will specifically and expressly state the exact phrases “means for” or “step for, and will also recite the word “function” (i.e., will state “means for performing the function of . . . ”), without also reciting in such phrases any structure, material or act in support of the function. Thus, even when the claims recite a “means for performing the function of . . . “or” step for performing the function of . . . ,” if the claims also recite any structure, material or acts in support of that means or step, or that perform the recited function, then it is the clear intention of the inventors not to invoke the provisions of 35 U.S.C. § 112 (f). Moreover, even if the provisions of 35 U.S.C. § 112 (f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

### BRIEF DESCRIPTION OF DRAWINGS

A more complete understanding of the present invention may be derived by referring to the detailed description when considered in connection with the following illustrative figures. In the figures, like reference numbers refer to like elements or acts throughout the figures.

FIG. 1 shows an isometric view of the amputation endcap device in accordance to one or more embodiments;

FIG. 2a shows a front view of the amputation endcap device in accordance to one or more embodiments;

FIG. 2b shows a cross-sectional view of FIG. 2a of the amputation endcap device in accordance to one or more embodiments;

FIG. 3 shows a top view of the amputation endcap device in accordance to one or more embodiments;

FIG. 4 shows a bottom view of amputation endcap device in accordance to one or more embodiments; and

FIG. 5 shows an isometric view of the amputation endcap device omitting the second amputation endcap device in accordance to one or more embodiments;

FIG. 6 shows an isometric view of the amputation endcap device of another embodiment in accordance to one or more embodiments; and

FIG. 7 shows a bottom isometric view of the amputation endcap device of another embodiment in accordance to one or more embodiments.

Elements and acts in the figures are illustrated for simplicity and have not necessarily been rendered according to any particular sequence or embodiment.

### DETAILED DESCRIPTION OF THE INVENTION

In the following description, and for the purposes of explanation, numerous specific details are set forth to provide a thorough understanding of the various aspects of the invention. It will be understood, however, by those skilled in the relevant arts, that the present invention may be practiced without these specific details. In other instances, known structures and devices are shown or discussed more generally to avoid obscuring the invention. In many cases, a description of the operation is sufficient to enable one to implement the various forms of the invention, particularly when the operation is to be implemented in software. It should be noted that there are many different and alternative configurations, devices, and technologies to which the disclosed inventions may be applied. The full scope of the inventions is not limited to the examples that are described below.

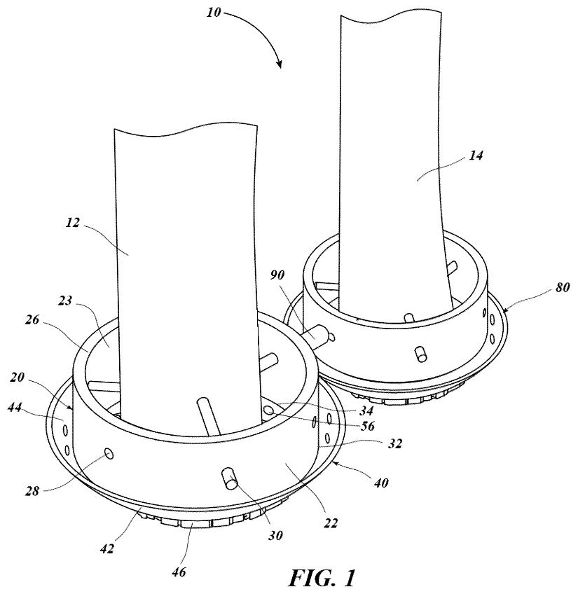

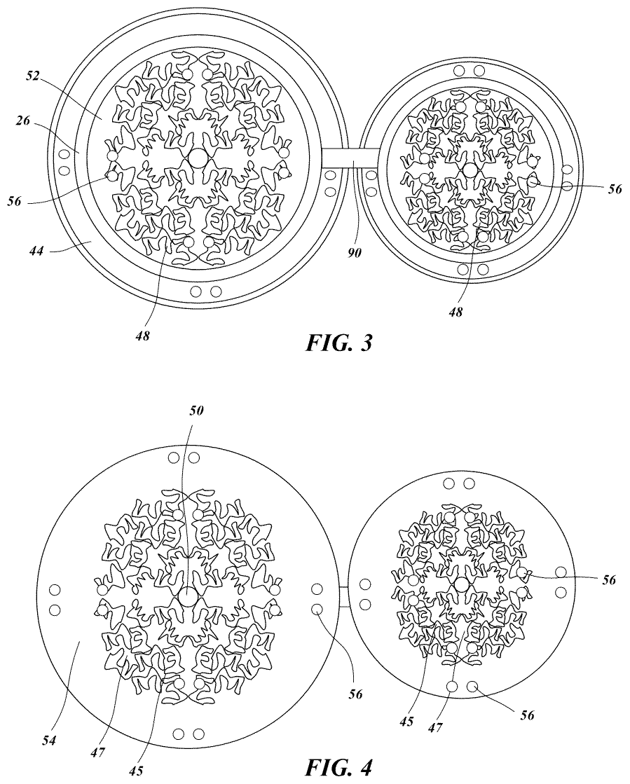

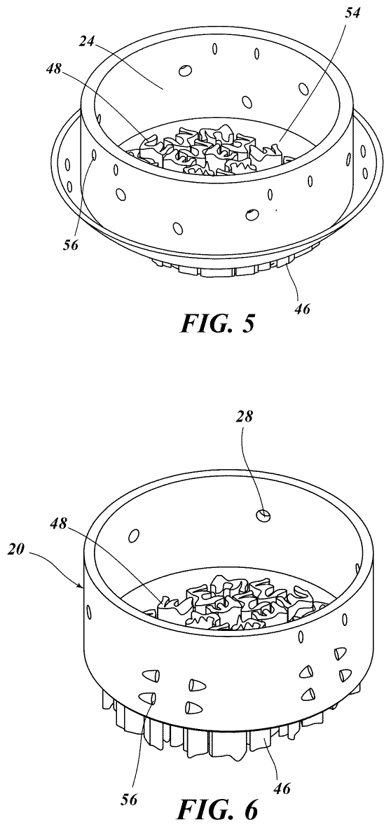

Referring to FIG. 1-5, a prosthetic endcap device is shown generally at 10. The prosthetic endcap device 10 for adult, pediatric or animal amputations of a patient having an amputated bone 12, 14. The endcap device comprises an annular body 20 having a longitudinal axis 21 and an interior cavity 24. The proximal end 26 of the annular body 20 can be configured to accommodate the amputated bone end, while the distal end 32 can define a bottom 34. The annular body 20 can extend from the proximal end 26 to the distal end 32 and can incorporate a concentric arrangement of an annular outer wall 22 and an annular inner wall 23, which together create a defined wall thickness between annular outer wall and the annular inner wall. The concentric arrangement of the annular outer wall 22 and the annular inner wall 23 creates a stress distribution system that reduces pressure points on the developing bone tissue.

In certain embodiments, the interior cavity 24 can have variable tolerance zones that accommodate both immediate fit requirements and anticipated growth patterns. In certain embodiments, the proximal end 26 can have a geometry that interfaces with the bone's natural contours while providing microscale surface variations that promote optimal tissue integration. The prosthetic endcap device 10 demonstrates can maintain its structural integrity through its concentric wall arrangement while allowing for natural bone remodeling. The bottom 34 at the distal end 32 can have an interior surface 52 and an exterior surface 54 wherein at least one of the interior surface and the exterior surface is a trabeculated surface. The trabeculated surface, can be an interior trabeculation 48 and an exterior trabeculation 46 can create a topography that promotes osseointegration and tissue attachment and can utilize both the interior and the exterior trabeculation to create a comprehensive environment for bone and tissue integration. In certain embodiments, the interior surface 52 can have longitudinal grooves that can be configured to enhance bone ingrowth.

In embodiments, the interior trabeculation 48 and the exterior trabeculation 46 can have at least one peaks 45 and at least one valley 47 wherein the trabeculated surface has a height and depth of between 1-2 millimeters. The trabeculated surface comprise of at least one peak 45 and at least one valley 47 can have interconnected microporous structures with 0.5-3.0 mm peak-to-valley heights and 30-70% surface porosity. In certain embodiments, the trabeculated surface can have load-bearing struts perpendicular to the surface and secondary cross-struts forming channels with 100-700 μm pore sizes and 20-80 μm surface roughness (Ra). Gradient porosity can be increased from base to surface, mimicking cancellous bone structure. The trabeculated surface can help with osteoblast attachment, vascular ingrowth, mechanical bone interlocking, and soft tissue adhesion.

In embodiments, the annular body 20 can be dimensioned to accommodate various length and sized bones including such as, for example, a femur, a fibula, a tibia, a humerus, a ulna, a radius or the like. The annular body's 20 annular inner wall 23 can be sized to provide multiple fitting configurations based on specific bone anatomy and surgical requirements which can be such as, for example, a loose fit arrangement allowing controlled movement between the bone end and interior surface, a press fit configuration providing direct contact pressure between the bone and device, and a calculated gap configuration maintaining a predetermined space between the amputated bone end and the annular inner wall. The gap dimension can be precisely controlled to accommodate such as, for example, post-surgical swelling, potential bone growth, insertion of biological, synthetic materials, or the like. The fitting configurations can be achieved through specific dimensioning of the interior cavity 24 relative to standard bone dimensions while accounting for surgical variations and patient-specific requirements. In certain embodiments, the annular inner wall 23 surface can be textured, smooth, trabeculated, or the like depending on the selected fitting configuration to optimize bone-device interface characteristics.

In embodiments, the annular body 20 can have a plurality of transosseous holes 28 positioned throughout the annular body 20, wherein the plurality of transosseous holes can accommodate transosseous screws 30 that can anchor the prosthetic endcap device 10 securely to the amputee's residual bone tissue. The plurality of transosseous holes 28 can be configured with varying angular orientations, including angled trajectories that enter the body at carefully calculated non-perpendicular approaches, straight pathways that traverse the structure perpendicularly, customized angles optimized for specific anatomical requirements, other surgical objectives, or the like. The spatial arrangement of the plurality of transosseous holes 28 demonstrates considerable flexibility in their positioning, allowing for offset patterns where the plurality of transosseous holes can be intentionally displaced from the central axis to maximize bone engagement, inline configurations that align multiple holes along a single reference plane for simplified surgical approaches, or staggered distributions that alternate hole positions to optimize load distribution and structural integrity.

In embodiments, the plurality of transosseous screws 30 can be optimized for secure skeletal fixation while minimizing bone trauma. The plurality of transosseous screws 30 can range from 0.5 to 5 millimeters in diameter, with a preferred range of 1 to 4 millimeters, and an optimal range of 2 to 3 millimeters. The plurality of transosseous screws 30 diameter selection balances several critical factors such as, for example, sufficient mechanical strength for load-bearing, minimal disruption of bone tissue, compatibility with standard surgical instrumentation, or the like. The plurality of transosseous screws 30 placement can preserve the central intramedullary canal and can be positioned to avoid penetration into this vital structure, maintaining the integrity of the bone marrow cavity, preservation of hematopoietic function, maintenance of normal bone metabolism, and retention of vascular supply pathways critical for bone healing and remodeling. The positioning of transosseous screws 30 around, rather than through, the intramedullary canal can maintain mechanical strength of the residual bone by avoiding stress concentrations in the central shaft which can support distributed load transfer between the prosthetic endcap device 10 and bone tissue while preserving anatomical structures essential for long-term bone health and prosthetic success. The plurality of transosseous screws 30 diameter ranges accommodate variations in patient bone quality, anatomical dimensions, and specific surgical requirements while maintaining consistent performance standards.

This adaptable of the plurality of transosseous holes 28 can provide surgeons with essential options for achieving optimal screw placement, ensuring robust skeletal fixation while accommodating diverse patient anatomies, varying bone qualities, and different surgical technique preferences. The versatility in both hole orientation and positioning patterns enables the prosthetic endcap device 10 to effectively address the unique challenges presented by individual cases, ultimately promoting successful osseointegration and long-term prosthetic stability. In other embodiments, the plurality of transosseous holes 28 can be omitted from the annular body 20 or can be a plurality of transosseous screws 30 that can thread into the plurality of transosseous holes 28 and into the bone 12.

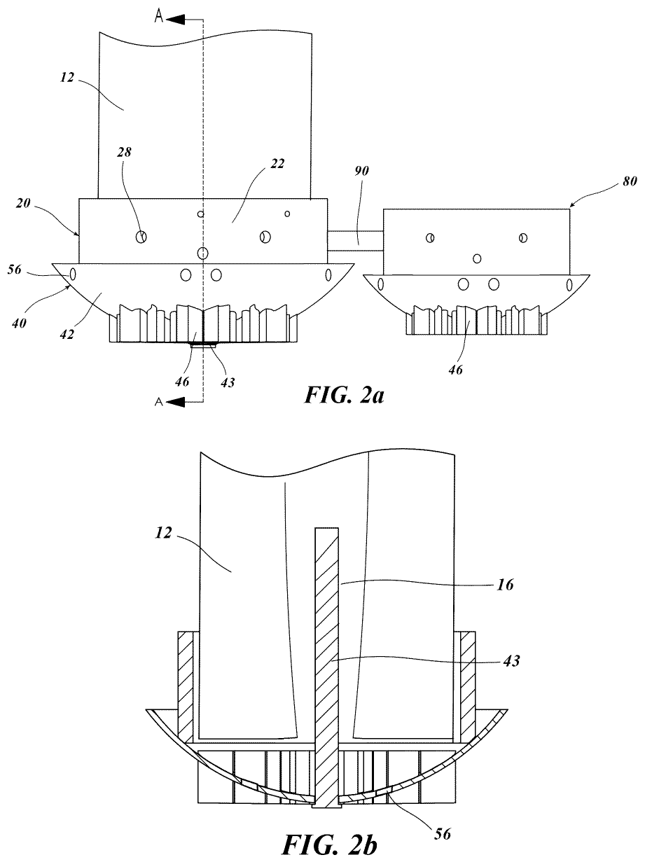

In embodiments, the prosthetic endcap device 10 can further comprise an expansion shield 40 that projects outwardly from the central axis at the distal end 32 of the annular body 20. The expansion shield 40 can have a width from such as, for example, at least 2 to 15 millimeters and more preferable 4-12 millimeters and even more preferable 5-10 millimeters. The expansion shield 40 can have a bulbous geometry but in other embodiments the expansion shield 40 can be any suitable shape and size based on specific anatomical requirements or surgical preferences and to allow for comfort for the user of a prothesis wherein the shape can be such as, for example, teardrop, ellipsoid, bell-shaped, tapered, curved, stepped configurations, or the like. In certain embodiments, the interior trabeculation 48 and the exterior trabeculation 46 can be the substantially the same shape as the expansion shield 40 and/or bottom 34 wherein the interior trabeculation and the exterior trabeculation can be at least one of a generally bulbous surface, a flat surface as shown in FIG. 2 or the interior trabeculation can be bulbous and the exterior trabeculation can be flat or any suitable shape to mate to the amputee's bone and a prosthetic.

The expansion shield 40 bulbous shape can provide functional advantages such as, for example, it creates a gradual transition zone between the prosthetic device and surrounding soft tissue, minimizes edge effects that could lead to tissue irritation or breakdown, and establishes a protective barrier against unwanted tissue migration. The expansion shield's 40 outward projection serves multiple functions such as, acting as a mechanical buffer, preventing direct contact between internal prosthetic components and adjacent soft tissues. The expansion shield 40 can have a smooth transition and rounded edges to prevent tissue irritation and promote optimal interface conditions between the device and surrounding anatomical structures. This attention to surface characteristics enhances long-term biocompatibility and supports stable soft tissue adaptation. The expansion shield 40 and the annular body 20 can be made from one piece or multiple pieces coupled together to form the prosthetic endcap device 10 and can be manufactured by such as, for example, 3D printing, molding, machined, or the like.

In embodiments, the annular body 20, the bottom 40 and/or the expansion shield 40 can further comprise a plurality of myodesis suture holes 56 designed to facilitate secure soft tissue reattachment to the prosthetic endcap device 10. The plurality of myodesis suture holes 56 can be uniformly distributed around the peripheral edge of the annular body, maintaining consistent inter-hole spacing to ensure balanced load distribution during and after surgical attachment wherein in other embodiments the plurality of myodesis holes can be randomly placed according to the doctor needs for suture placement. In embodiments, the plurality of myodesis suture holes 56 can have a diameter of such as, for example, at least 0.25 millimeters to 5 millimeters and more particularly 1 millimeter to 4 millimeters and even more particularly 1 millimeter to 2 millimeters, wherein the plurality of myodesis suture holes can accommodate standard surgical suture sizes while maintaining structural integrity of the device material. The plurality of myodesis suture holes 56 spacing patterns can serve multiple critical functions such as, for example, it enables surgeons to achieve symmetric tissue reattachment, preventing irregular tension patterns that could compromise healing or lead to tissue breakdown, and the spacing facilitates standardized surgical protocols, allowing consistent suture placement regardless of the specific anatomical variations encountered.

The plurality of myodesis suture holes 56 can enable a surgeon to securely anchor residual muscle tissue to the prosthetic endcap device 10 using a circumferential suturing technique wherein the even distribution of myodesis suture holes can support uniform tension across the muscle-device interface, critical for preventing stress concentrations that could lead to suture pullout or tissue damage. The uniform tension distribution can promote optimal healing conditions and helps maintain proper muscle length-tension relationships in the residual limb. In certain embodiments, the plurality of myodesis suture holes 56 can have smoothly contoured edges to minimize suture abrasion and reduce the risk of suture failure.

In embodiments, the bottom 34 of the device can comprise an intramedullary hole 50, configured to accommodate an intramedullary screw 43 wherein the intramedullary screw can go through both the interior surface 52 and exterior surface 54 of the bottom 34 and can be positioned to fastened to the end of the amputated bone. The intramedullary hole 50 and intramedullary screw 43 can be the primary anchor point, working in conjunction with the trabeculated surfaces to establish a stable bone-implant interface wherein the intramedullary screw can have a diameter of such as, for example, at least 1 millimeter to 8 millimeter, and more preferable 1.5 millimeter to 5 millimeter and even more preferable 3 millimeter to 4 millimeter. The intramedullary hole 50 can be positioned either centrally or in a slightly off-center configuration within the prosthetic endcap device 10 wherein the positioning flexibility enables surgeons to optimize screw placement based on individual patient anatomy and surgical requirements wherein the intramedullary screw 43 can be placed in the intramedullary space 16 of the bone 12. The off-center option particularly allows for strategic positioning that avoids interference with critical neurovascular structures while maintaining secure fixation within the bone marrow cavity. The geometric relationship between the intramedullary hole 50 and surrounding trabeculated regions can distribute mechanical loads effectively while promoting biological fixation, bone and tissue growth.

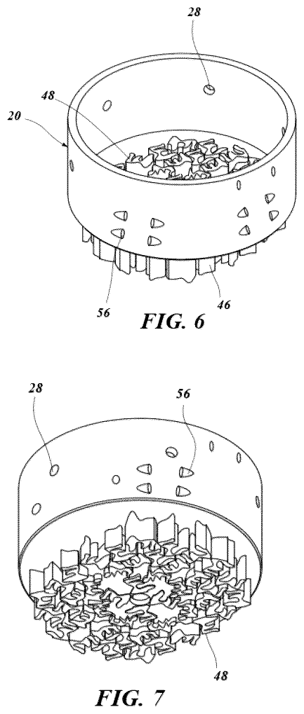

In embodiments, the expansion shield 40 can extend radially beyond the trabeculated surface by such as, for example, at least 1 millimeters-20 millimeters, and more preferably 2-14 millimeters, and still even more preferably 5 millimeters to 10 millimeters. The expansion shield 40 and the bottom 34 can be the same shape creating a generally bulbous profile or in other embodiments the shape can be such as, for example, teardrop, ellipsoid, bell-shaped, tapered, curved, stepped configurations, or the like. The expansion shield 40 and the bottom 34 can be one piece or multiple pieces. The expansion shield 40 can support soft tissue without constraining growth of the amputated bone. In other embodiments, the expansion shield 40 can be omitted and the annular body 20 can have a bottom 34 with the trabeculated surfaces as shown in FIG. 6 and FIG. 7.

In certain embodiments, a second prosthetic endcap device 80 can be coupled to the prosthetic endcap device 10 by at least one connection rod 90 and can be sized and positioned to hold a second amputated bone 14, as shown in FIG. 1. The connection rod 90 linking the two endcap devices can be implemented either as a telescoping mechanism or as a fixed-length rod. The telescoping rod can provide a dynamic length adjustment capability to accommodate bone growth, particularly crucial in adult and pediatric applications or cases where bone lengthening is anticipated wherein the fixed-rod can offer maximum stability for cases where bone growth accommodation may not be required.

The dimensional differential between the primary endcap device 10 and the second endcap device 80 can match the natural anatomical size relationship between paired bones, such as the radius and ulna or tibia and fibula wherein the size optimization ensures proper biomechanical function while maintaining anatomical alignment. The coupling interface between the connection rod 90 and both endcap devices can provide a secure attachment while allowing for necessary adjustments during surgical implementation. This interface incorporates features that facilitate proper alignment and stable fixation while maintaining the ability to accommodate anatomical variations.

In certain embodiments, the prosthetic endcap device 10 incorporates radiopaque markers strategically positioned at predetermined locations on the annular body to facilitate precise radiographic visualization during surgical placement and post-operative monitoring. The radiopaque markers serve multiple critical functions in the surgical implementation and long-term monitoring of the device which can enable accurate intraoperative positioning by providing clear radiographic reference points for device orientation and depth of insertion relative to the amputated bone. The markers also facilitate post-operative assessment of device position and stability through standard radiographic imaging protocols. The markers can be manufactured from biocompatible materials with high atomic numbers, such as tantalum or platinum, ensuring optimal visibility under various imaging modalities while maintaining device biocompatibility.

The markers size and distribution pattern can provide unambiguous reference points without interfering with the device's mechanical properties or biological integration capabilities. The predetermined positioning of these markers can correspond to critical anatomical landmarks and device features, enabling surgeons to verify proper alignment with bone structures and soft tissue interfaces wherein the alignment verification capability is particularly crucial during initial device placement to ensure optimal positioning for long-term functional outcomes. Each marker can be permanently integrated into the device structure during manufacturing, ensuring reliable long-term radiographic visualization for ongoing clinical assessment and monitoring of device position and stability throughout the implant lifetime.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

Certain embodiments are described herein, including the best mode known to the inventors for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

We claim:

1. A prosthetic endcap device for amputations of a patient having amputated bone, the amputated bone having a bone end, the endcap device comprising:
an annular body having an axis, an interior, a proximal end configured to accommodate the bone end and a distal end defining a bottom;
an expansion shield extending outwardly from the axis at the distal end of the annular body;
a plurality of transosseous holes in the annular body, each transosseous hole configured to accommodate a transosseous screw;
wherein the bottom comprises:
an intramedullary hole configured to accommodate an intramedullary screw;
an interior surface; and
an exterior surface;
wherein at least one of the interior surface and the exterior surface is a trabeculated surface.

2. The prosthetic endcap device of claim 1, wherein a second prosthetic endcap device is coupled to the prosthetic endcap device by at least one connection rod and is sized and positioned to hold a second amputated bone.

3. The prosthetic endcap device of claim 1, wherein the trabeculated surface has at least one peaks and at least one valley wherein the trabeculated surface has a height and depth of between 1-2 millimeters.

4. The prosthetic endcap device of claim 1, wherein the expansion shield extends radially beyond the trabeculated surface by 5-10 millimeters.

5. The prosthetic endcap device of claim 1, wherein the annular body has a thickness of 3-5 millimeters and a height of 20% of a total height of the endcap device.

6. The prosthetic endcap device of claim 1, wherein the plurality of transosseous holes have a diameter of 1-2 millimeters and are spaced around the periphery.

7. The prosthetic endcap device of claim 1, wherein the intramedullary hole is configured to receive the intramedullary screw having a diameter of 2-6 millimeters.

8. The prosthetic endcap device of claim 7, wherein the intramedullary screw is positioned to be fastened to the end of the amputated bone.

9. The prosthetic endcap device of claim 1, wherein annular body is sized to be either press fit or loose fit onto the amputated bone.

10. The prosthetic endcap device of claim 1, wherein the plurality of transosseous holes are placed to avoid a central intramedullary canal of the amputees bone.

11. The prosthetic endcap device of claim 1, wherein the annular body and expansion shield comprise a biocompatible metal selected from the group consisting of PEEK, nylon, carbon fiber, titanium, titanium alloys, and stainless steel.

12. The prosthetic endcap device of claim 4, wherein the expansion shield extends at an angle between 10-45 degrees relative to the axis and has a bulbous shape.

13. The prosthetic endcap device of claim 5, wherein the annular body has a height between 15-40 millimeters.

14. The prosthetic endcap device of claim 2, wherein the connection rod comprises a telescoping mechanism or a fixed rod to accommodate bone growth.

15. The prosthetic endcap device of claim 2, wherein the second prosthetic endcap device is substantially smaller than the prosthetic endcap device to accommodate a smaller amputated bone.

16. The prosthetic endcap device of claim 1, wherein the plurality of transosseous holes are arranged in at least one circular patterns at different heights along the annular body.

17. The prosthetic endcap device of claim 1, further comprising radiopaque markers positioned at predetermined locations on the annular body.

18. The prosthetic endcap device of claim 1, wherein the interior surface comprises longitudinal grooves configured to enhance bone ingrowth.

19. The prosthetic endcap device of claim 1, wherein annular body has a plurality of myodesis holes for suture.
